(19) United States
(12) Patent Application Publication
(10) Pub. No.: US 2006/0122241 A1
(43) Pub. Date: Jun. 8, 2006

(54) Novel macrocycles for the treatment of cancer diseases

(76) Inventor: Gerhard Hoefle, Braunschweig (DE)

Correspondence Address: Ronald R Santucci;Frommer Lawrence & Haug, 745 Fifth Avenue, New York, NY, 10151, US

(21) Appl. No.: 10/520,766

(22) PCT Filed: Jul. 15, 2003
(86) PCT No.: PCT/EP03/07663

§ 371 Date: Jul. 22, 2005

(30) Foreign Application Priority Data
Jul. 15, 2002 (DE) 102 32 094.2

(51) Int. Cl. A61K 31/427; C07D 417/02

(52) U.S. Cl. 514/365; 548/181

(57) ABSTRACT

The present invention relates to novel macrocycles of the general formula (I) and to the use thereof in the treatment of cancer diseases.

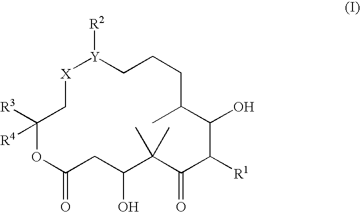

Epothilones (DE 4 138 042) are natural substances having extraordinary biological action, for example as mitosis inhibitors, microtubuli-modifying agents, cytotoxic agents or fungicides. In particular, they have properties similar to paclitaxel and in addition have an activity superior to that of paclitaxel (Taxol®) in some tests. A number of derivatives are currently in clinical studies for the treatment of cancer diseases (Nicolaou et al. in Angew. Chem. Int. Ed. 1998, 37, 2014-2045; Florsheimer et al. in Expert Opin. Ther. Patents 2001, 11, 951-968).

The aim of the present invention was to provide novel epothilone-type derivatives that are more effective and have better pharmacological properties than the natural substances and the hitherto known derivatives.

The present invention relates to compounds of the general formula (I): 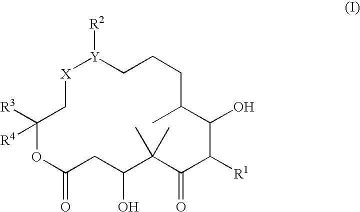
wherein
R¹ is a C₁₋₆alkyl, a C₂₋₆alkynyl or a C₂₋₆alkenyl radical,
R² is a hydrogen atom or a C₁₋₆alkyl radical,
X—Y is selected from the following groups: 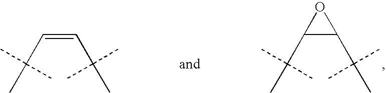
R³ is a halogen atom or a C₁₋₆alkyl, a C₂₋₆alkenyl or a C₁₋₆-heteroalkyl radical,
R⁴ is a bicycloaryl radical, a bicycloheteroaryl radical or a group of formula —C(R⁵)═CHR⁶,
R⁵ is a hydrogen atom or a methyl group and
R⁶ is an optionally substituted aryl or heteroaryl group,
or a pharmacologically acceptable salt, solvate, hydrate or a pharmacologically acceptable formulation thereof.

The term “alkyl” denotes a saturated, straight-chain or branched hydrocarbon group having from 1 to 20 carbon atoms, especially from 1 to 12 carbon atoms and more especially from 1 to 6 carbon atoms, for example a methyl, ethyl, propyl, isopropyl, isobutyl, tert-butyl, n-hexyl, 2,2-dimethylbutyl or n-octyl group.

The terms “alkenyl” and “alkynyl” denote at least partially unsaturated, straight-chain or branched hydrocarbon groups having from 2 to 20 carbon atoms, especially from 2 to 12 carbon atoms and more especially from 2 to 6 carbon atoms, for example an ethenyl, allyl, acetylenyl, propargyl, isoprenyl or hex-2-enyl group. Preferably, alkenyl groups have one or two double bonds, especially one double bond, and alkynyl groups have one or two triple bonds, especially one triple bond.

In addition, the terms “alkyl”, “alkenyl” and “alkynyl” denote groups in which one or more hydrogen atoms have been replaced by a halogen atom (preferably F or Cl), for example a 2,2,2-trichloroethyl or a trifluoromethyl group.

The term “heteroalkyl” denotes an alkyl, alkenyl or alkynyl group in which one or more (preferably 1, 2 or 3) carbon atoms have each been replaced by an oxygen, nitrogen, phosphorus, boron, selenium, silicon or sulphur atom (preferably an oxygen, sulphur or nitrogen atom). The term “heteroalkyl” furthermore denotes a carboxylic acid or a group derived from a carboxylic acid, such as, for example, acyl, acylalkyl, alkoxycarbonyl, acyloxy, acyloxyalkyl, carboxyalkylamide or alkoxycarbonyloxy.

Examples of heteroalkyl groups include groups of the formulae R^{a}—O—Y^{a}—, R^{a}—S—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—O—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein Ra is a hydrogen atom, a C₁-C₆alkyl group, a C₂-C₆alkenyl group or a C₂-C₆alkynyl group; R^{b} is a hydrogen atom, a C₁-C₆alkyl group, a C₂-C₆alkenyl group or a C₂-C₆alkynyl group; R^{c} is a hydrogen atom, a C₁-C₆alkyl group, a C₂-C₆alkenyl group or a C₂-C₆alkynyl group; R^{d} is a hydrogen atom, a C₁-C₆-alkyl group, a C₂-C₆alkenyl group or a C₂-C₆-alkynyl group and Y^{a} is a direct bond, a C₁-C₆alkylene group, a C₂-C₆alkenylene group or a C₂-C₆alkynylene group, wherein each heteroalkyl group contains at least one carbon atom, and one or more hydrogen atoms may each have been replaced by fluorine or chlorine atoms. Specific examples of heteroalkyl groups are methoxy, trifluoromethoxy, ethoxy, n-propoxy, isopropoxy, tert-butoxy, methoxymethyl, ethoxymethyl, methoxyethyl, methylamino, ethylamino, dimethylamino, diethylamino, isopropylethylamino, methylaminomethyl, ethylaminomethyl, di-isopropylaminoethyl, enol ethers, dimethylaminomethyl, dimethylaminoethyl, acetyl, propionyl, butyryloxy, acetoxy, methoxycarbonyl, ethoxycarbonyl, N-ethyl-N-methylcarbamoyl and N-methylcarbamoyl. Further examples of heteroalkyl groups are nitrile, isonitrile, cyanate, thiocyanate, isocyanate, isothiocyanate and alkylnitrile groups.

The term “cycloalkyl” denotes a saturated or partially unsaturated (e.g. cycloalkenyl) cyclic group having one or more rings (preferably 1 or 2 rings) that form a skeleton containing from 3 to 14 carbon atoms, preferably from 3 to 10 (especially 3, 4, 5, 6 or 7) carbon atoms. The term “cycloalkyl” furthermore denotes groups in which one or more hydrogen atoms have each been replaced by fluorine, chlorine, bromine or iodine atoms or by OH, ═O, SH, ═S, NH₂, ═NH or NO₂ groups, for example cyclic ketones, such as, for example, cyclohexanone, 2-cyclohexenone or cyclopentanone. Further specific examples of cycloalkyl groups are cyclopropyl, cyclobutyl, cyclopentyl, spiro[4,5]decanyl, norbornyl, cyclohexyl, cyclopentenyl, cyclohexadienyl, decalinyl, cubanyl, bicyclo[4.3.0]nonyl, tetralinyl, cyclopentylcyclohexyl, fluorocyclohexyl and the cyclohex-2-enyl group.

The term “heterocycloalkyl” denotes a cycloalkyl group as defined above in which one or more (preferably 1, 2 or 3) ring carbon atoms have each been replaced by an oxygen, nitrogen, silicon, selenium, phosphorus or sulphur atom (preferably an oxygen, sulphur or nitrogen atom). Preferably, a heterocycloalkyl group has 1 or 2 rings containing from 3 to 10 (especially 3, 4, 5, 6 or 7) ring atoms. The term “heterocycloalkyl” furthermore denotes groups in which one or more hydrogen atoms have each been replaced by fluorine, chlorine, bromine or iodine atoms or by OH, ═O, SH, ═S, NH₂, ═NH or NO₂ groups. Examples are the groups piperidyl, morpholinyl, urotropinyl, pyrrolidinyl, tetrahydrothiophenyl, tetrahydropyranyl, tetrahydrofuryl, oxacyclopropyl, azacyclopropyl and 2-pyrazolinyl and also lactams, lactones, cyclic imides and cyclic anhydrides.

The term “alkylcycloalkyl” denotes groups that, corresponding to the above definitions, contain both cycloalkyl and alkyl, alkenyl or alkynyl groups, for example alkylcycloalkyl, alkylcycloalkenyl, alkenylcycloalkyl and alkynylcycloalkyl groups. Preferably, an alkylcycloalkyl group contains a cycloalkyl group having one or two ring systems that form a skeleton containing from 3 to 10 (especially 3, 4, 5, 6 or 7) carbon atoms and one or two alkyl, alkenyl or alkynyl groups containing 1 carbon atom or from 2 to 6 carbon atoms.

The term “heteroalkylcycloalkyl” denotes alkylcycloalkyl groups as defined above in which one or more (preferably 1, 2 or 3) carbon atoms have each been replaced by an oxygen, nitrogen, silicon, selenium, phosphorus or sulphur atom (preferably an oxygen, sulphur or nitrogen atom). Preferably, a heteroalkylcycloalkyl group contains 1 or 2 ring systems having from 3 to 10 (especially 3, 4, 5, 6 or 7) ring atoms and one or two alkyl, alkenyl, alkynyl or heteroalkyl groups containing 1 or from 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The term “aryl” or “Ar” denotes an aromatic group that has one or more rings, such as, for example, bicycloaryl, and is formed by a skeleton that contains from 6 to 14 carbon atoms, preferably from 6 to 10 (especially 6) carbon atoms. The term “aryl” (or “Ar”) furthermore denotes groups in which one or more hydrogen atoms have each been replaced by fluorine, chlorine, bromine or iodine atoms or by OH, SH, NH₂ or NO₂ groups. Examples are the groups phenyl, naphthyl, biphenyl, 2-fluorophenyl, anilinyl, 3-nitrophenyl and 4-hydroxyphenyl.

The term “heteroaryl” denotes an aromatic group that has one or more rings, such as, for example, bicycloheteroaryl, and is formed by a skeleton that contains from 5 to 14 ring atoms, preferably from 5 to 10 (especially 5 or 6) ring atoms, and contains one or more (preferably 1, 2, 3 or 4) oxygen, nitrogen, phosphorus and/or sulphur ring atoms (preferably O, S and/or N). The term “heteroaryl” furthermore denotes groups in which one or more hydrogen atoms have each been replaced by fluorine, chlorine, bromine or iodine atoms or by OH, SH, NH₂ or NO₂ groups. Examples are 4-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl groups.

The term “aralkyl” denotes groups that, corresponding to the above definitions, contain both aryl and alkyl, alkenyl, alkynyl and/or cycloalkyl groups, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetralin, dihydronaphthalenes, indanone, phenylcyclopentyl, cumene, cyclohexylphenyl, fluorene and indan. Preferably, an aralkyl group has one or two aromatic ring systems (1 or 2 rings) containing from 6 to 10 carbon atoms and one or two alkyl, alkenyl and/or alkynyl groups containing 1 or from 2 to 6 carbon atoms and/or a cycloalkyl group containing 5 or 6 ring carbon atoms.

The term “heteroaralkyl” denotes an aralkyl group as defined above in which one or more (preferably 1, 2, 3 or 4) carbon atoms have each been replaced by an oxygen, nitrogen, silicon, selenium, phosphorus, boron or sulphur atom (preferably an oxygen, sulphur or nitrogen atom), that is to say, groups that, corresponding to the above definitions, contain both aryl or heteroaryl and alkyl, alkenyl, alkynyl and/or heteroalkyl and/or cycloalkyl and/or heterocycloalkyl groups. Preferably, a heteroaralkyl group has one or two aromatic ring systems (1 or 2 rings) containing 5 or from 6 to 10 carbon atoms, and one or two alkyl, alkenyl and/or alkynyl groups containing 1 or from 2 to 6 carbon atoms and/or a cycloalkyl group containing 5 or 6 ring carbon atoms, wherein 1, 2, 3 or 4 of those carbon atoms have each been replaced by oxygen, sulphur or nitrogen atoms.

Examples are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, heteroarylheterocycloalkenyl, heteroarylalkylcycloalkyl, heteroarylalkylheterocycloalkenyl, heteroarylheteroalkylcycloalkyl, heteroarylheteroalkylcycloalkenyl and heteroarylheteroalkylheterocycloalkyl groups, wherein the cyclic groups are saturated or mono-, di- or tri-unsaturated. Specific examples are the groups tetrahydroisoquinolinyl, benzoyl, 2- or 3-ethylindolyl, 4-methylpyridino, 2-, 3- or 4-methoxyphenyl, 4-ethoxyphenyl and 2-, 3- or 4-carboxyphenylalkyl.

The terms “cycloalkyl”, “heterocycloalkyl”, “alkylcycloalkyl”, “heteroalkylcycloalkyl”, “aryl”, “heteroaryl”, “aralkyl” and “heteroaralkyl” denote groups in which one or more hydrogen atoms of such groups have each been replaced by fluorine, chlorine, bromine or iodine atoms or by OH, ═O, SH, ═S, NH₂, ═NH or NO₂ groups.

The expression “optionally substituted” denotes groups in which one or more hydrogen atoms have each been replaced by fluorine, chlorine, bromine or iodine atoms or by OH, ═O, SH, ═S, NH₂, ═NH or NO₂ groups. This expression also denotes groups that are substituted by unsubstituted C₁-C₆alkyl, C₂-C₆alkenyl, C₂-C₆alkynyl, C₁-C₆heteroalkyl, C₃-C₁₀cycloalkyl, C₂-C₉heterocycloalkyl, C₆-C₁₀aryl, C₁-C₉heteroaryl, C₇-C₁₂aralkyl or C₂-C₁₁-heteroaralkyl groups.

Protecting groups are known to the person skilled in the art and are described, for example, in P. J. Kocienski, Protecting Groups, Georg Thieme Verlag, Stuttgart, 1994 and in T. W. Greene, P. G. M. Wuts, Protective Groups in Organic Synthesis, John Wiley & Sons, New York, 1999. Common amino-protecting groups are, for example, tert-butoxycarbonyl (Boc), benzyloxycarbonyl (Cbz, Z), benzyl (Bn), benzoyl (Bz), fluorenylmethoxycarbonyl (Fmoc), allyloxycarbonyl (Alloc), trichloroethoxycarbonyl (Troc), acetyl and trifluoroacetyl groups.

Compounds of formula (I) may, as a result of their substitution, contain one or more centres of chirality. The present invention accordingly includes not only all pure enantiomers and all pure diastereoisomers but also mixtures thereof in any mixing ratio. Also covered by the present invention are all cis/trans isomers of the compounds of the general formula (I) as well as mixtures thereof. The present invention furthermore includes all tautomeric forms of the compounds of formula (I).

Preference is given to compounds of formula (I) wherein R¹ is a methyl group.

Preference is given furthermore to compounds of formula (I) wherein R² is a hydrogen atom or a methyl group.

Preference is given also to compounds of formula (I) wherein R³ is a methyl group, a trifluoromethyl group or a group of formula COOH (especially a trifluoromethyl group).

Preference is given in addition to compounds of formula (I) wherein R⁶ is an optionally substituted 5- or 6-membered heteroaryl radical having 1, 2 or 3 hetero atoms selected from S, N and O.

R⁶ is especially preferably an optionally substituted thiazole ring or pyridine ring.

For the radicals or terms “bicycloaryl” and “bicycloheteroaryl” reference may also be made to the following prior art:
1. K. C. Nicolaou et al. Chemistry & Biology 7, 593 (2000)
2. K.-H. Altmann et al. Bioorg. Med. Chem. Lett. 10, 2765 (2000)
3. K. C. Nicolaou et al. Chem. Eur. J. 6, 2783 (2000)

Also preferably, R⁴ is a group of the following formula:

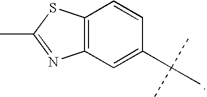

Examples of pharmacologically acceptable salts of the compounds of formula (I) are salts (or mixed salts) of physiologically acceptable mineral acids, such as hydrochloric acid, sulphuric acid and phosphoric acid, or salts of organic acids, such as methanesulphonic acid, p-toluenesulphonic acid, lactic acid, acetic acid, trifluoroacetic acid, citric acid, succinic acid, fumaric acid, maleic acid and salicylic acid. Compounds of formula (I) may be solvated, especially hydrated. The hydration may occur, for example, during the preparation process or as a consequence of the hygroscopic nature of the initially anhydrous compounds of formula (I). When the compounds of formula (I) contain asymmetric carbon atoms, they may either be in the form of achiral compounds, diastereoisomeric mixtures or mixtures of enantiomers or in the form of optically pure compounds. Also included in the present invention are all cis/trans isomers of the present compounds of the general formula (I) and mixtures thereof.

The pharmaceutical compositions according to the present invention contain at least one compound of formula (I) as active ingredient and optionally one or more carriers and/or one or more adjuvants.

The pro-drugs (see, for example, R. B. Silverman, Medizinische Chemie, VCH Weinheim, 1995, chapter 8, pages 361ff), to which the present invention also relates, consist of a compound of formula (I) and at least one pharmacologically acceptable protecting group, which is split off under physiological conditions, for example an alkoxy, aralkyloxy, acyl or acyloxy group, such as, for example, an ethoxy, benzyloxy, acetyl or acetoxy group.

Likewise included within the scope of the present invention is the therapeutic use of the compounds of formula (I), their pharmacologically acceptable salts or solvates and hydrates, and formulations and pharmaceutical compositions.

The present invention relates also to the use of those active ingredients in the preparation of medicaments for the treatment of cancer diseases. Generally, compounds of formula (I) are administered either individually or combined with any other desired therapeutic agent using known and acceptable methods. Such therapeutically useful agents can be administered by one of the following routes: orally, for example in the form of dragees, coated tablets, pills, semi-solids, soft or hard capsules, solutions, emulsions or suspension; parenterally, for example in the form of injectable solutions; rectally, in the form of suppositories; by inhalation, for example in the form of a powder formulation or spray, transdermally or intra-nasally. For the preparation of such tablets, pills, semi-solids, coated tablets, dragées and hard gelatin capsules, the therapeutically usable product can be mixed with pharmacologically inert, inorganic or organic carriers, for example lactose, sucrose, glucose, gelatin, malt, silica gel, starch or derivatives thereof, talc, stearic acid or its salts, dried skimmed milk and the like. For the production of soft capsules, carriers such as, for example, vegetable oils, liquid paraffin, animal or synthetic oils, wax, fat and/or polyols can be used. For the preparation of liquid solutions and syrups, carriers such as, for example, water, alcohols, aqueous salt solution, aqueous dextroses, polyols, glycerol, vegetable oils, liquid paraffin, animal and/or synthetic oils can be used. For suppositories, carriers such as, for example, vegetable oils, liquid paraffin, animal and/or synthetic oils, wax, fat and/or polyols may be used. For aerosol formulations, compressed gases suitable for that purpose such as, for example, oxygen, nitrogen, noble gases and/or carbon dioxide, may be used. The pharmaceutically usable agents may also contain additives for preservation, for stabilisation, emulsifiers, sweeteners, flavourings, salts for modifying the osmotic pressure, buffers, encapsulating additives and/or antioxidants.

Combinations with other therapeutic agents may contain further active ingredients usually used for the treatment of cancer diseases.

For the treatment of cancer diseases, the dose of the biologically active compound according to the invention can vary within wide limits and can be adjusted according to individual requirement. A dose of from 1 μg to 100 mg/kg body weight per day is generally suitable, with a dose of from 10 μg to 25 mg/kg per day being preferred. In appropriate cases, the dose may also be lower or higher than the values given above.

The epothilone derivatives of formula (I) can be prepared from known compounds (WO 0232844) using standard reactions (Nicolaou et al. Angew. Chem. Int. Ed. 1998, 37, 2014-2045) in accordance with the following scheme.

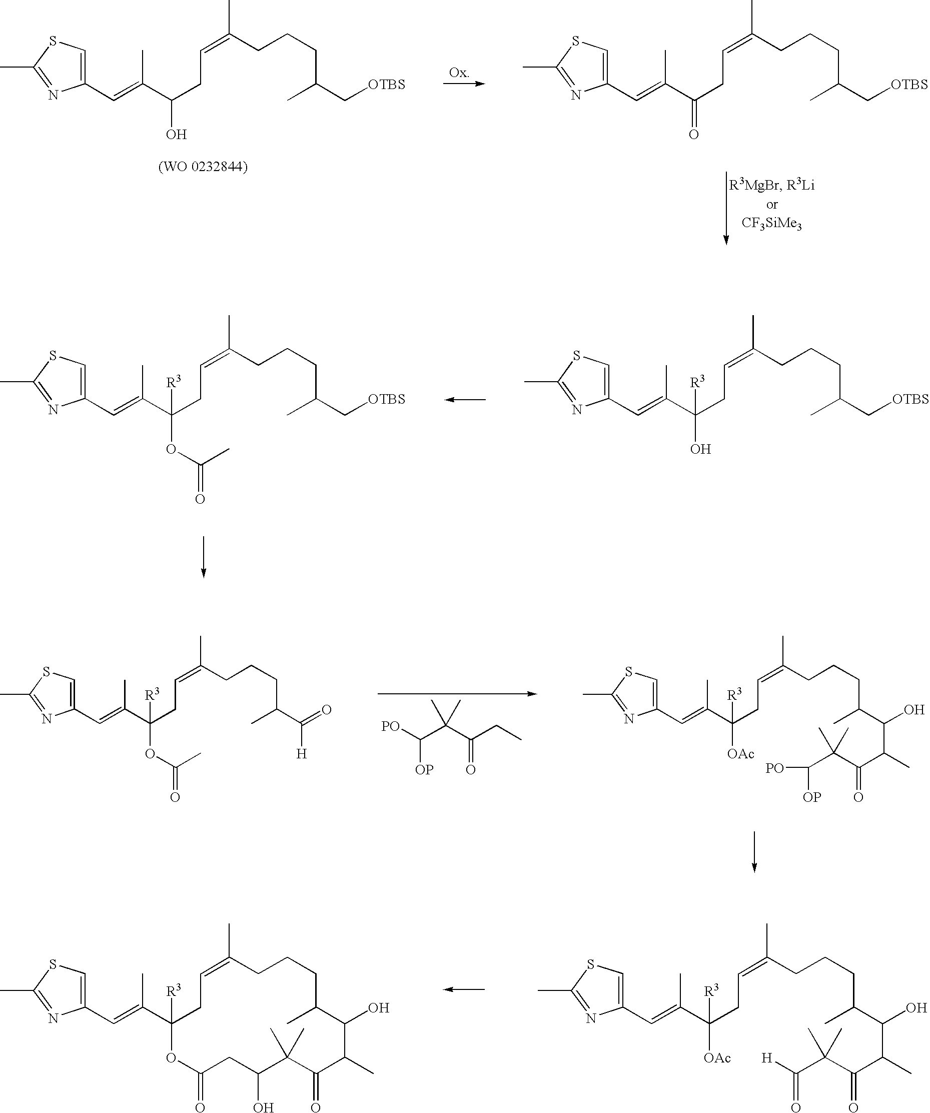

In this scheme, P is a standard protecting group for aldehydes, such as, for example, methyl, or two groups P together are a CH₂CH₂ group.

Compounds in which R⁴ is bicycloheteroaryl can be prepared by way of the following intermediate steps:

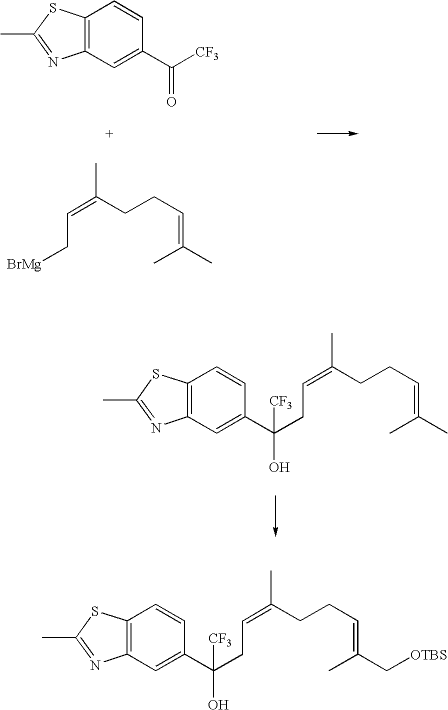

The further synthesis follows the known epothilone syntheses.

### EXAMPLES

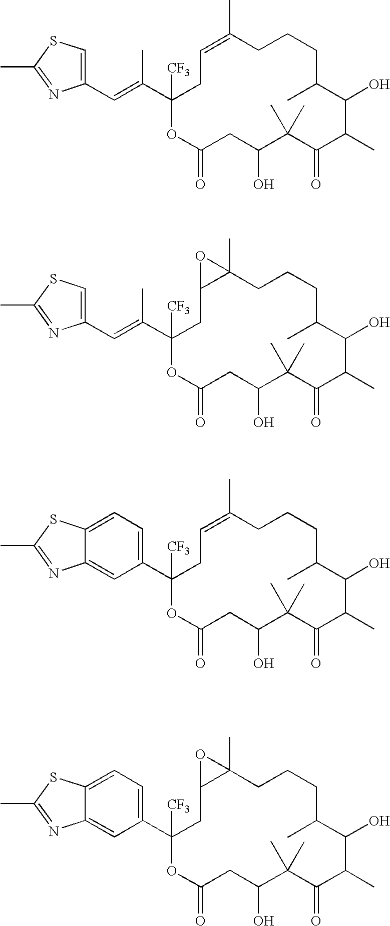

1. Compounds of the general formula (I): 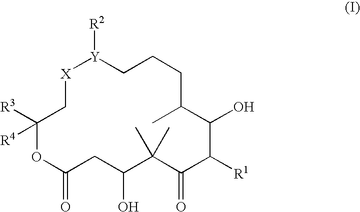
wherein
R¹ is a C₁₋₆alkyl, a C₂₋₆alkynyl or a C₂₋₆alkenyl radical,
R² is a hydrogen atom or a C₁₋₆alkyl radical,
X—Y is selected from the following groups: 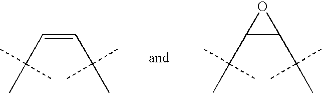
R³ is a halogen atom or a C₁₋₆alkyl, a C₂₋₆alkenyl or a C₁₋₆heteroalkyl radical,
R⁴ is a bicycloaryl radical, a bicycloheteroaryl radical or a group of formula —C(R⁵)═CHR⁶,
R⁵ is a hydrogen atom or a methyl group and
R⁶ is an optionally substituted aryl or heteroaryl group,
or a pharmacologically acceptable salt, solvate, hydrate or a pharmacologically acceptable formulation thereof.

2. Compounds of formula (I), wherein R¹ is a methyl group.

3. Compounds according to claim 1, wherein R² is a hydrogen atom or a methyl group.

4. Compounds according to claim 1, wherein R³ is a methyl group, a trifluoromethyl group or a group of formula COOH.

5. Compounds according to claim 1, wherein R³ is a trifluoromethyl group.

6. Compounds according to claim 1, wherein R⁶ is an optionally substituted 5- or 6-membered heteroaryl radical having 1, 2 or 3 hetero atoms selected from S, N and O.

7. Compounds according to claim 1, wherein R⁶ is an optionally substituted thiazole ring or pyridine ring.

8. Compounds according to claim 1, wherein R⁴ is a group having the following formula:

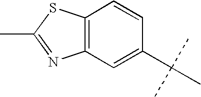

9. Pharmaceutical composition, which contains a compound according to claim 1 and optionally one or more carriers and/or one or more adjuvants.

10. Use of a compound or a pharmaceutical composition according to claim 1 in the treatment of cancer diseases.
